(19) United States
(12) Patent Application Publication
(10) Pub. No.: US 2003/0096866 A1
(43) Pub. Date: May 22, 2003

(54) Methods for activating lipid catabolism and improving lipid metabolism in small intestinal epithelium

(75) Inventors: Tadashi Hase, Haga-gun (JP); Takatoshi Murase, Haga-gun (JP); Hiroyuki Watanabe, Haga-gun (JP); Hidehiko Kondo, Haga-gun (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Kao Corporation, TOKYO (JP)

(21) Appl. No.: 10/131,188

(22) Filed: Apr. 25, 2002

(30) Foreign Application Priority Data
Apr. 26, 2001 (JP) 2001-129847

(51) Int. Cl. A61K 31/231

(52) U.S. Cl. 514/547

(57) ABSTRACT

Disclosed are a method for activating lipid metabolism in the small intestine epithelium and also a method for promoting accumulation of fatty acids into the small intestine epithelium, each of which features administering an effective amount of a diglyceride. Ingestion of the diglyceride leads to accumulation of the fatty acids in the small intestine. The fatty acids so accumulated promote induction of β-oxidation, thereby not only activating lipid catabolism but also making it difficult to allow lipids to accumulate as triglycerides. This series of actions eventually results in development of lowering action for blood remnant-like lipoprotein level and also lowering action for blood leptin level, and hence, lipid metabolism is improved. Further, energy consumption is enhanced by promoting the induction of β-oxidation and activating lipid catabolism.

### BACKGROUND OF THE INVENTION

a) Field of the Invention

This invention relates to a method for promoting accumulation of fatty acids into the small intestinal epithelium, and also to a method for improving lipid metabolism in the small intestine epithelium for the suppression of triglyceride synthesis, the enhancement of β-oxidation, the enhancement of uncoupling protein (UCP) expression, the promotion of energy consumption, the lowering of blood leptin level, the lowering of blood remnant level and/or the like purpose.

b) Description of the Related Art

From research in recent years, elucidations have been made increasingly as to a connection between lipid metabolism disorders, such as an increase in blood leptin level and an increase in blood remnant level, and diseases such as angina pectoris, myocardial infarction, cerebral thrombosis, cerebral infarction and aortic aneurysm.

It is, therefore, desired to lower the remnant and leptin levels by improving lipid metabolism (Fertil Steril March 2002; 77(3), 433-44).

Remnant-like lipoprotein particles (RLP; called “remnant particles” or simply “remnant”) have been reported to be a strong risk factor for the above-described diseases, because they are susceptible to absorption into blood vessel walls and cholesterol in RLP so absorbed accumulates in the blood vessel walls. Leptin, which is a hormone secreted mainly from adipose tissues, on the other hand, has been reported to perform control on body fat and serum lipids by promoting energy consumption, through burning promoting effect for body fat. If lipid metabolism disorders continue, however, the serum leptin level increases and leptin can no longer exhibit its inherent effect. If this situation arises, it is necessary to lower the serum leptin level such that leptin can smoothly perform its function.

It is, therefore, very important for the prevention and treatment of diseases, which are associated with lipid metabolism, to lower blood remnant level and blood leptin level and also to promote energy consumption.

In general, lipids (triglycerides) ingested as a meal are degraded by lipase into fatty acids and 2-monoglyceride in the small intestine, and subsequently, most of the fatty acids and 2-monoglycerides are usually resynthesized into the triglycerides in the small intestine epithelium, followed by a move into blood. A portion of the fatty acids so formed, on the other hand, is subjected to catabolism in the small intestine epithelium and is converted into energy. In other words, the energy of the fatty acids is converted into an electrochemical potential of protons within mitochondria through a series of pathways such as β-oxidation and electron transport systems.

It is a function of an uncoupling protein (UCP) to uncouple oxidative phosphorylation. Described specifically, the electron transport system and ATP synthesis are closely coupled with each other by a proton gradient across mitochondrial inner membranes, and UCP is a special channel which eliminates this proton gradient in a short-cut manner. When UCP is activated, chemical energy of an oxidized substrate is converted into heat instead of being employed for ATP synthesis (“Seikagaku (Biochemistry)”, 70, 212-216, 1998; “Rinsho Kagaku (Clinical Science)”, 34, 1043-1048, 1998). Accordingly, a functional disorder of UCP and lowering in its expression are considered to decrease energy consumption and to lead to accumulation of energy and obesity. Conversely, an increase in the expression of UCP and its activation are considered to increase energy consumption and to result in anti-obesity.

It is also known that the small intestine is a tissue active in the expression of UCP, that the expression of small intestine UCP varies depending on dietary lipids, and that the expression of small intestine UCP is increased especially by fish oil having lipid metabolism improving effect. In view of these, small intestine UCP is suggested to play an important role in lipid metabolism (Biochem J., 345, 161-179, 2000; Biochimica et Biophysica Acta, 1530, 15-22, 2001).

The β-oxidation system is a principal metabolic degradation pathway for fatty acids. A group of enzymes, such as MCAD (medium-chain acyl-CoA dehydrogenase) and ACO (acyl-CoA oxidase), play parts in the β-oxidation pathway. The β-oxidation system plays an important role not only in the degradation of fatty acids but also in thermogenesis through conversion of fatty acids into energy. Deficit of β-oxidation enzyme has been reported to lead to a reduction in energy expenditure (J. Clin. Invest., 102, 1724-1731, 1998). Therefore, enhancement of β-oxidation is considered to improve lipid metabolism and energy metabolism and also to lead to an improvement in hyperleptinemia.

PPAR (peroxisome proliferator activated receptor) is a transcription factor which controls development of UCP and β-oxidation related molecules (acyl-CoA oxidase, medium chain acyl CoA dehydrogenase, etc.). Participation of fatty acids in the activation of PPAR is known well from experiments on cell level. As has been described above, oil (triglycerides) is generally degraded into 2-monoglycerides and fatty acids in the small intestinal tract, and subsequent to absorption, the 2-monoglycerides and fatty acids are resynthesized into triglycerides in the small intestine epithelium. The present inventors, therefore, postulated that, if it is possible to cause fatty acids to accumulate in the small intestine epithelium and to increase its concentration there, level/expression of β-oxidation related molecules and UCP would be increased. Under this postulation, the present inventors have proceeded with research. No specific method has been proposed yet to date for storing fatty acids in cells.

### SUMMARY OF THE INVENTION

An object of the present invention is to provide a method for promoting accumulation of fatty acids into the small intestine epithelium. Another object of the present invention is to provide a method for improving lipid metabolism for the suppression of triglyceride synthesis, the enhancement of β-oxidation, the enhancement of uncoupling protein (UCP) expression, the promotion of energy consumption, the lowering of blood leptin level, the lowering of blood remnant level and/or the like purpose. A further object of the present invention is to provide a method for activating lipid catabolism in the small intestine.

Diglycerides are used for foods, as they have unique functions without side effects. Specifically, cholesterol level lowering agents (JP 63-104917 A), body weight gain suppressants (JP 4-300826 A), general-purpose oil compositions (U.S. Pat. No. 6,004,611), oil or fat compositions (WO 01/13733), vegetable-sterol-containing oil or fat compositions (WO 99/48378), body fat burning promoters (JP 2001-64672 A), and the like have been proposed.

Nonetheless, absolutely nothing is known as to what effects diglycerides exhibit in the small intestine.

The present inventor, therefore, conducted various investigations with a view to elucidating effects of diglycerides in the small intestine, especially in the small intestine epithelium. As a result, it has been found that diglycerides are degraded in the cavity of the small intestinal tract and subsequent to absorption in the small intestine epithelium, the resulting fatty acids are hardly reconstituted into triglycerides and accumulated there, and also that the thus-formed fatty acids induce the expression of genes involved in lipid metabolism in the S.I. and suppress synthesis of triglycerides.

In other words, the present inventors have found that diglycerides have the lipid catabolism activating effect in the small intestine and lipid metabolism improving effect.

In one aspect of the present invention, there is thus provided a method for activating lipid catabolism in the small intestine epithelium, which comprises administering an effective amount of a diglyceride.

In another aspect of the present invention, there is also provided a method for promoting accumulation of fatty acids into the small intestine epithelium, which comprises administering an effective amount of a diglyceride.

In a further aspect of the present invention, there is also provided a method for inducing expression of a small intestine lipid metabolic gene, which comprises administering an effective amount of a diglyceride.

In a still further aspect of the present invention, there is also provided a method for suppressing synthesis of a triglyceride in the small intestine epithelium, which comprises administering an effective amount of a diglyceride.

In a yet further aspect of the present invention, there is also provided a method for promoting energy consumption, which comprises administering an effective amount of a diglyceride.

Ingestion of diglycerides results in the accumulation of fatty acids in the small intestine. The fatty acids so accumulated promote induction of a β-oxidation enzyme to activate lipid catabolism at the small intestine, so that energy consumption is promoted and the fatty acids are hardly resynthesized into triglycerides. Further, ingestion of diglycerides over an extended time promotes burning of not only the diglycerides but also triglycerides ingested through other meals, and therefore, accumulation of body fat is suppressed. In addition, blood remnant-like lipoprotein and leptin levels are lowered, and lipid metabolism is improved.

### BRIEF DESCRIPTION OF THE DRAWINGS

FIG. 1 shows variations in energy consumptions by rats, which ingested diglycerides (DAG) and triglycerides (TAG), respectively, in 22 hours;

FIG. 2 illustrates variations in the concentrations of ¹³C—CO₂ in expirations from mice, which ingested diglycerides(DAG) and triglycerides (TAG), respectively, after administration of ¹³C-tripalmitin; and

FIGS. 3A and 3B depict percent accumulations of epididymal fat and percent accumulations of mesenteric fat in mice which ingested diglycerides(DAG) and triglycerides (TAG), respectively.

### DETAILED DESCRIPTION OF THE INVENTION AND PREFERRED EMBODIMENTS

Constituent fatty acids of the diglyceride for use in the present invention may preferably be those having carbon numbers of from 8 to 24, especially from 16 to 22. Among the entire constituent fatty acids of the diglyceride, the content of unsaturated fatty acids may be preferably from 70 to 100 wt. % (hereinafter described simply as “%”), more preferably from 90 to 100%, particularly preferably from 93 to 100%, most preferably from 95 to 100%. From the standpoint of further enhancing the lipid metabolism improving effect and the fatty acid accumulation promoting effect, the (cis-form unsaturated)/(trans-form unsaturated+saturated) ratio may be preferably 6 or greater, more preferably from 9 to 25, still more preferably from 9 to 20. On the other hand, the particularly preferred content of the trans-form unsaturated fatty acids in the diglyceride may be 5% or lower and the especially preferred content of the saturated fatty acids may also be 5% or lower. From the standpoint of effects and oxidation stability, 15 to 90% of the constituent fatty acids comprise ω3 unsaturated fatty acids, with 20 to 80% being more preferred, 30 to 70% being still more preferred, and 40 to 65% being particularly preferred. Examples of the ω3 unsaturated fatty acids can include α-linolenic acid (C18:3), stearidonic acid (C18:4), eicosapentaenoic acid (C20:5), docosapentaenoic acid (C22:5) and docosahexaenoic acid (C22:6), with α-linolenic acid, eicosapentaenoic acid and docosahexaenoic acid being preferred, and α-linolenic acid being more preferred. Diglycerides include 1,3-diglycerides and 1,2-diglycerides (2,3-diglycerides). More preferably, the weight ratio of the 1,3-diglycerides to the 1,2-diglycerides may be 7:3. From the stand point of enhancing the lipid metabolism improving effect, increasing the accumulation of fatty acids and improving the industrial productivity, the 1,3-diglycerides may amount preferably to 50% or more, more preferably to 55 to 100%, especially to 60 to 90% of the whole diglycerides.

The diglyceride for use in the present invention can be produced, for example, by subjecting an oil or fat, which contains target constituent fatty acids, and glycerol to transesterification or by causing lipase to act on a mixture of the target constituent fatty acids or esters thereof and glycerol to conduct transesterification. From the standpoint of avoiding isomerization during the reaction, the transesterification making use of lipase is more preferred. In this transesterification making use of lipase, it is preferred, for the prevention of isomerization during a purification stage after completion of the reaction, to conduct the purification under such mild conditions that no isomerization of fatty acids would take place.

As is appreciated from the foregoing, it is preferred to use the diglyceride as an oil or fat composition which also contains triglycerides and the like. From the standpoint of effects, the oil or fat composition may contain preferably 15 to 100%, more preferably 40 to 99%, particularly preferably 60 to 95%, most preferably 80 to 95% of diglycerides. Fatty acids formed from diglycerides as a result of degradation by lipase in the course of digestion are more prone to accumulate in the small intestine epithelium than those formed from triglycerides. Use of an oil or fat composition containing 15% or more of diglycerides can, therefore, bring about excellent lipid metabolism improving effect.

The oil or fat composition may contain triglycerides. From the standpoint of effects, taste and flavor, and oxidation stability, the content of the triglycerides in the oil or fat composition may range from 0 to 85%, preferably from 1 to 59.9%, more preferably from 5to 39.9%, most preferably from 5to 19.9%. As constituent fatty acids of the triglyceride, unsaturated fatty acids having the carbon numbers of which range from 16 to 22 may be contained preferably in a proportion of from 55 to 100%, more preferably in a proportion of from 70 to 100%, especially in a proportion of from 80 to 100%, most preferably in a proportion of from 90 to 97%, from the standpoint of effects, taste and flavor, and texture. From the standpoint of oxidation stability, ω3 unsaturated fatty acids may also be contained, as constituent fatty acids of triglycerides, preferably in a proportion of from 0 to 40%, more preferably in a proportion of from 0 to 30%, particularly in a proportion of from 0 to 20%, most preferably from 0 to 15%.

The oil or fat composition may also contain monoglycerides. From the standpoint of taste and flavor and oxidation stability, their content may be 0 to 30%, preferably 0.1 to 10%, more preferably 0.1 to 5%, especially preferably 0.1 to 2%, most preferably 0.1 to 1.5%. Constituent fatty acids of the monoglycerides may preferably the same as those of the diglycerides from the viewpoint of manufacture.

Free fatty acids contained in the oil or fat composition have an offensive taste and from the standpoint of taste, their content may be set below 10%, preferably below 5%, more preferably below 2.5%, especially preferably below 1%, most preferably below 0.5%.

Preferably, an antioxidant can be added to the oil or fat composition to improve its oxidation stability. Examples of the antioxidant can include butylhydroxyanisole (BHA), butylhydroxytoluene (BHT), vitamin A, vitamin C, vitamin E, phospholipids, polyphenol, and tert-butylhydroquinone (TBHQ). Two or more of these antioxidants can also be used in combination. The antioxidant may be contained preferably in a proportion of from 0.005 to 0.2%, especially in a proportion of from 0.04 to 0.1% in the oil or fat composition.

It is also preferred to further add a crystallization inhibitor to the oil or fat composition. Examples of crystallization inhibitors usable in the present invention can include polyol fatty acid esters such as polyglycerol condensed licinoleic acid esters, polyglycerol fatty acid esters, sucrose fatty acid esters, sorbitan fatty acid esters, polyoxyethylene sorbitan fatty acid esters and propylene glycol fatty acid esters. Two or more of these crystallization inhibitors can also be used in combination.

The crystallization inhibitor may be contained preferably in a proportion of from 0.02 to 0.5%, more preferably from 0.05 to 0.2% in the oil or fat composition.

In vegetable oil, phytosterols are contained in a proportion of from 0.05 to 1.2% or so. However, the content of phytosterols in an oil or fat composition differs depending on its production process. Use of a fatty acid, which is available abundantly on the market and was obtained by distillation, as a raw material, for example, results in an oil or fat composition in which the content of a phytosterol is low. In such a case, it is preferred to add phytosterols such that the oil or fat composition contains phytosterols in a total proportion of from 0.05 to 20%, especially from 0.3 to 1.2%. Examples of such phytosterols can include free forms such as α-sitosterol, β-sitosterol, stigmasterol, campesterol, α-sitostanol, β-sitostanol, stigmastanol and campestanol; and ester forms such as their fatty acid esters, ferulic acid esters and cinnamic acid esters.

In the methods according to the present invention, the diglyceride can be administered preferably at a daily dosage in a range of from 0.1 to 25 g, especially from 0.1 to 10 g per adult, generally once to several times in a day. Administration of 0.1 g/day in terms of the diglyceride is essential for the development of the effects.

When the methods according to the present invention are applied for the prevention or treatment of a disease, illustrative dosage forms can include oral preparations, for example, solid preparations such as powders, granules, capsules, pills and tables, and liquid preparations such as solutions, suspensions and emulsions. These oral preparations can each be produced by adding, in addition to the above-described oil or fat composition, one or more of excipients, disintegrators, binders, lubricants, surfactants, alcohols, water, water-soluble polymers, sweeteners, corrigents, sour agents, and the like, which are commonly employed depending on the forms of oral preparations. When the oil or fat composition is used, its content in each orally administered pharmaceutical may range generally from 0.05 to 100%, with 1 to 50% being particularly preferred, although the content varies depending on the application purpose and preparation form of the medicine.

To ingest diglycerides in the form of foods in the methods according to the present invention, processed oil or fat foods containing the glycerides can be used. For example, glycerides can be formulated into health foods, functional foods, dietary foods or the like, which exhibit specific functions to promote health. Specific examples can include capsules, tables and granules; bakery foods such as breads, cakes, cookies, pies and bakery mixes; dressings such as French dressing; oil-in-water emulsion foods such as mayonnaise; water-in-oil emulsion foods such as margarine and spreads; confectioneries such as creams, chocolate and potato chips, ice cream and dessert; drinks; sauces; coffee whitener; whipped cream; barbecue sauce; peanut butter; frying shortening; baking shortening; processed meat products; frozen foods; and food materials such as cooking oils useful for tempura, fries and frizzled dishes. These foods can each be produced by adding, in addition to an oil or fat composition, one or more food materials commonly employed depending on the kind of the food. The content of the oil or fat composition in each of these foods may range generally from 0.05 to 100%, particularly preferably from 0.5 to 80%, although it differs depending on the kind of the food.

Administration of diglycerides or an oil or fat composition with diglycerides contained therein accelerates the accumulation of fatty acids in the small intestine epithelium. Further, the expression of the gene of β-oxidation enzymes and the gene of UCP, each of which takes part in the metabolism of lipids in the small intestine, is promoted. Furthermore, the synthesis of triglycerides in the small intestine epithelium is suppressed.

By these effects for promoting the accumulation of fatty acids in the small intestine epithelium and activating lipid catabolism, energy consumption is enhanced. Further, continued ingestion of diglyceride or an oil or fat composition with diglyceride contained therein facilitates burning not only of the diglyceride itself but also of lipids ingested as meals and also suppresses their accumulation as body fat.

In addition, as a result of activation of lipid metabolism in the small intestine epithelium by the methods of the present invention, PLLP-C, which is determined by quantitating blood RLP with cholesterol (Nakajima, K., Clin. Chim. Acta, 223,53-71, 1993), and blood leptin level are lowered.

Examples will hereinafter be described. It is, however, to be borne in mind that the present invention shall not be limited the following Examples.

The following oil compositions were prepared in accordance with the below-described procedure.

Oil or Fat Composition A

Fatty acids, which had been obtained by hydrolyzing commercial soybean oil the trans acid content of which was 0.8%, were subjected to wintering to lower the content of saturated fatty acids. Using commercial immobilized 1,3-position-selective lipase (“Lipozyme 3A”, trade name; product of Novo-Nordisk Industries A.S.) as a catalyst, those fatty acids and glycerol were subjected to esterification at 40° C. After the lipase preparation was filtered off, the reactant was purified by molecular distillation to obtain an oil or fat composition A.

Oil or Fat Composition B

Fatty acids, which had been obtained by hydrolyzing commercial rapeseed oil the trans acid content of which was 0.6%, and glycerol were subjected to esterification at 40° C. by using “Lipozyme 3A”. After the lipase preparation was filtered off, the reactant was purified by molecular distillation to obtain an oil or fat composition B.

Oil or Fat Composition C

Fatty acids, which had been obtained by hydrolyzing commercial rapeseed oil the trans acid content of which was 2.8%, and glycerol were subjected to esterification at 40° C. by using “Lipozyme 3A”. After the lipase preparation was filtered off, the reactant was purified by molecular distillation to obtain an oil or fat composition C.

Oil or Fat Composition D

Commercial high docosahexaenoic acid oil and glycerol were mixed together, and subjected to transesterification at 100° C. under reduced pressure by using an alkali catalyst (sodium methoxide). After the catalyst was filtered off, the reactant was purified by molecular distillation to obtain an oil or fat composition D.

Oil or Fat Composition E

Linseed oil fatty acids and glycerol were subjected to esterification at 40° C. by using “Lipozyme IM” (trade name; product of Novo-Nordisk Industries A.S.). After the lipase preparation was filtered off, molecular distillation was conducted at 215° C. Subsequent to water washing, deodorization was performed at 215° C. for 2 hours to obtain an oil or fat composition E.

The glyceride compositions and diglyceride-constituent fatty acid compositions of the thus-produced oil or fat compositions (A-E) and soybean oil were analyzed by the below-described methods. The results are shown in Tables 1 and 2.

[Determination of the Glyceride Compositions]

Each oil was trimethyl silylated with a silylating agent (“Silylating Agent TH”, trade name; product of Kanto Kagaku K.K.), and using a capillary column (“DBTM-1”, trade name; product of J & W Scientific Inc.), the trimethyl silylated oil was then analyzed by gas chromatography.

[Determination of the Diglyceride-Constituent Fatty Acid Compositions]

Diacylglycerol fractions in each oil were collected by column chromatography [after triglyceride fractions had been eluted using “Wako Gel C-200”, trade name; product of Wako Pure Chemical Industries, Ltd.) and hexane, the diacylglycerol fractions were obtained with a 70:30 mixed solvent of hexane and ethyl ether]. Subsequent to methyl esterification by a method known per se in the art, an analysis was performed by gas chromatography equipped with a capillary column (“CP-SIL88”, trade name; product of Chrompack Inc.).

TABLE 1
Glyceride Compositions

| Oil or fat | Mono- | Diglycerides | | |
|---|---|---|---|---|
| composition | glycerides | (% of 1,3-DG) | Triglycerides | Phytosterols |
| A | 1.1 | 85.7 (59.9) | 12.7 | 0.5 |
| B | 0.9 | 85.0 (59.5) | 13.2 | 0.9 |
| C | 1.5 | 80.8 (56.5) | 16.7 | 1.0 |
| D | 0.9 | 53.1 (37.0) | 45.8 | 0.2 |
| E | 1.0 | 84.8 (59.3) | 14.0 | 0.2 |
| Soybean oil | ND | 1.0 | 98.7 | 0.3 |

ND: Not detected

TABLE 2
Fatty Acid Compositions (%)

| | Oil or fat composition | | | | | Commerical |
|---|---|---|---|---|---|---|
| Constituent fatty acids | A | B | C | D | E | soybean oil |
| C14 | — | — | — | 1.6 | — | — |
| C16 | 1.3 | 3.8 | 4.2 | 9.3 | 5.3 | 10.8 |
| C16:1 | — | — | — | 3.4 | — | — |
| C18 | 1.2 | 2.8 | 1.7 | 2.7 | 3.3 | 4.2 |
| C18:1 | 26.9 | 65.2 | 58.0 | 11.0 | 18.7 | 24.4 |
| Cis | 26.9 | 65.2 | 56.3 | NT | 18.7 | 24.4 |
| Trans | 0.0 | 0.0 | 1.7 | NT | — | 0.0 |
| C18:2 | 60.7 | 17.8 | 24.3 | 1.4 | 15.4 | 51.6 |
| Cis | 59.7 | 17.4 | 21.0 | NT | 15.4 | 51.3 |
| Trans | 1.0 | 0.4 | 3.3 | NT | — | 0.3 |
| C18:3 | 7.8 | 9.0 | 8.7 | 0.7 | 55.2 | 7.2 |
| Cis | 6.6 | 6.7 | 7.1 | NT | 52.8 | 6.7 |
| Trans | 1.2 | 1.2 | 1.6 | NT | 2.4 | 0.5 |
| C20 | 0.0 | 0.5 | 1.2 | — | — | 0.4 |
| C20:1 | — | — | — | 1.6 | — | — |
| C20:5 | — | — | — | 6.6 | — | — |
| C22:1 | — | — | — | 1.1 | — | — |
| C22:6 | — | — | — | 45.7 | — | — |
| Uk | 1.0 | 2.0 | 1.9 | 14.9 | 0.8 | 1.4 |
| Trans | 2.2 | 1.6 | 6.6 | NT | 2.4 | 0.8 |
| Saturated | 2.5 | 7.1 | 7.1 | 13.6 | 8.6 | 15.4 |
| Trans + saturated | 4.7 | 8.7 | 13.7 | — | 11.0 | 16.2 |
| Cis | 94.3 | 89.3 | 84.4 | — | 86.9 | 82.4 |
| Cis/(trans + saturated) | 20.1 | 11.3 | 6.2 | NT | 7.9 | 5.1 |

### EXAMPLE 1

### Small Intestine Perfusion Test

The following test was conducted in accordance with the method described in J. Lipid Res., 39, 963 (1998).

Under anesthesia, Wistar rats (male, 6 weeks old) were each incised at the abdomen, and a cannula (“PE50”, trade name; product of Clay Adams, Inc.) was arranged right underneath the pylorus. By a restraint gauge, an emulsion of triglycerides or diglycerides (triglycerides of diglycerides calculated as fatty acids: 90 mM, sodium chloride: 0.15M, 10 mM tris-HCl buffer: q.s. to pH 7.0, taurocholic acid: 10 mM) was perfused at a rate of 4.5 mL/hr (Experiment 1). Five hours later, the perfusing was stopped, and 1 mL of RI-labeled fatty acids was promptly injected together with the emulsion of triglycerides or diglycerides (Experiment 2). Namely, Experiment 1 was conducted such that the final concentration of [carboxy-¹⁴C]TO (triolein) or 1,3-[carboxy-¹⁴C]DO (diolein) reached 3.2×10⁶ dpm/mL, while Experiment 2 was conducted such that the final concentration of [-¹⁴C]linoleic acid reached 1.6×10⁶ dpm/mL. Subsequently, the above-described emulsion of triglycerides or diglycerides was injected again at the rate of 4.5 mL/hr. Five minutes later, Nembutal was injected into the abdominal cavity, the small intestine (40 cm from the pylorus) was sampled and placed in ice-cold 0.15 M sodium chloride. It took 5 minutes from the completion of the injection of the labeled substance until the sampling of the small intestine in the ice-cold saline. After the small intestine was cut into four equal parts and were then opened, the small intestine was washed with 0.15 M sodium chloride (once), 0.2% Triton-X100 (once), and 0.15 M sodium chloride (twice). The mucosa of the small intestine was scraped off and homogenized by a glass/Teflon® homogenizer in 0.15 M sodium chloride (10 mL). From 1 mL of the mucosa homogenate, lipids were extracted by the Folch partition method. The thus-obtained lipids were developed on a TLC plate (hexane:diethyl ether:acetic acid=80:20:1 (v/v/v, chloroform: acetone=96:4 (v/v), and measurements were conducted to determine the quantities of the label absorbed in FFA, 1,3-diglycerides, 1,2-diglycerides and triglycerides, respectively. The test results are shown in Table 3.

TABLE 3

| | Triglycerides | Diglycerides | Significant test |
|---|---|---|---|
| Free fatty acids | 100 | 182 | <0.05 |
| 1,3-Diglycerides | 100 | 400 | <0.001 |
| 1,2-Diglycerides | 100 | 106 | — |
| Triglycerides | 100 | 94 | <0.001 |

Shown in terms of relative value when the amounts of the fatty acids and respective glycerides existed during the perfusing of triglycerides were each supposed to be 100.

When the diglycerides were perfused, the amounts of free acids and 1,3-diglycerides existed in the mucosa of the small intestine epithelium were significantly high compared with the corresponding amounts when the triglycerides were perfused. On the other hand, no significant difference was observed in the amount of the 1,2-diglyceride. In the diglyceride-administered group, the amount of triglycerides occurred as a result of re-synthesis in the small intestine epithelium was significantly lower compared with that in the triglyceride administration group.

### EXAMPLE 2

### Induction of Small Intestine Lipid Metabolic Gene Expression by the Ingestion of Diglycerides

Wistar rats (male, 7 weeks old) were each fed with an experimental feed with 20% of a diglyceride-containing oil composition or soybean oil contained therein and reared for 7 days. On the last day, those rats were each dissected to sample the tissue of the small intestine. From the tissue of the small intestine, RNA was isolated, and by Northern blotting, the expressed quantity of a lipid metabolism associated (β-oxidation) enzyme (MCAD: medium-chain acyl-CoA dehydrogenase) mRNA was analyzed. The results are shown in Table 4.

TABLE 4

| | Soybean | Oil or fat | Oil or fat |
|---|---|---|---|
| | oil | composition B | composition E |
| MCAD mRNA | 100 | 130 | 145 |

Shown in terms of relative value when the expressed quantity of the MCAD mRNA in the case of soybeans was supposed to be 100.

By the ingestion of the diglyceride-containing oil or fat compositions B or E, the expression of the small intestine lipid metabolic gene was promoted, and lipid metabolism was enhanced. Further, the diglyceride containing α-linolenic acid as a main constituent fatty acid activated the lipid metabolism system more strongly than the diglyceride containing linoleic acid or oleic acid as a main constituent fatty acid.

### EXAMPLE 3

### Inhibition Test of Triglyceride Synthesis in the Small Intestine Epithelium

Using FCS (fetal calf serum)-free, Dulbecco's modified Eagle's medium (D-MEM) with 5% FBS and 70 μg/mL kanamycin added therein, a rat small-intestine epithelial cell strain, IEC-6, was incubated under 5% CO₂ at 37° C. Individual fatty acids (oleic acid, linoleic acid, γ-linolenic acid, arachidonic acid, α-linolenic acid, eicosapentaenoic acid, and docosahexaenoic acid) were formed into complexes with 250 μM fatty-acid-free bovine serum albumin, and were added at a concentration of 200 μM, respectively. Twenty-four hours later, the individual cultures were washed with PBS and subsequent to treatment with tripsin, were peeled off from Culture dishes. Those cultures were separately suspended in portions of HBSS which contained Nile Red (100 ng/mL). Subsequent to incubation at room temperature for 5 minutes or longer, FACS analysis was conducted. From average fluorescence intensities, synthesized quantities of triglycerides were measured. The results are shown in Table 5.

TABLE 5

| Fatty acid | | Fluorescence intensity |
|---|---|---|
| Linoleic acid | ω6 | 100 |
| γ-Linolenic acid | | 70 |
| Arachidonic acid | | 87 |
| Oleic acid | ω9 | 191 |
| α-Linolenic acid | ω3 | 42 |
| Eicosapentaenoic acid | | 52 |
| Docosahexaenoic acid | | 49 |

Relative values of average fluorescence intensities when the average fluorescence intensity of linoleic acid was supposed to be 100.

As a result, among these fatty acids, those most hardly synthesized into triglycerides were the ω3 fatty acids (α-linolenic acid, eicosapentaenoic acid and docosahexaenoic acid), followed by the ω6 fatty acids (linoleic acid, γ-linolenic acid and arachidonic acid). The ω9 fatty acid (oleic acid) was most liable to synthesis into the corresponding triglyceride among these fatty acids.

It has been found that a difference arises in the amount of synthesized triglycerides depending on the kinds of fatty acids which exist in the epithelial cells of the small intestine.

### EXAMPLE 4

### Effect of Diglycerides on Energy Metabolism

Male rats of an SD strain (7 weeks old) (Japan Charles River Inc.) were provided, and they were provisionally reared for 3 days. Using a 10% diglyceride (DAG) added feed (DAG group: n=6) or a 10% triglyceride (TAG) added feed (TAG group: n=7), they were then subjected for 1 week to two-meals-a-day rearing (eating time: 8:00 to 9:00, 21:00 to 22:00) in which the feed was given twice a day. With respect to the rats which had learned the timing of feed ingestion as described above, an expiration analysis was conducted for 22 hours (19:00 to 17:00). Using “Oxymax v. 5. 61” (trade name; manufactured by Columbus Instruments), the expiration analysis was conducted to measure the volume of oxygen consumed by the rats and the volume of carbon dioxide excreted by the rats.

TABLE 6
Compositions of Rat Feeds

| | TAG feed group | DAG feed group |
|---|---|---|
| | (%) | (%) |
| TAG | 10 | 0 |
| DAG | 0 | 10 |
| Casein | 20 | 20 |
| Cellulose | 8.1 | 8.1 |
| Mineral mix | 4 | 4 |
| Vitamin mix | 2.2 | 2.2 |
| Potato starch | 55.5 | 55.5 |
| L-methionine | 0.2 | 0.2 |
| Total | 100.0 | 100.0 |

DAG: Oil or fat composition B
TAG: Soybean oil

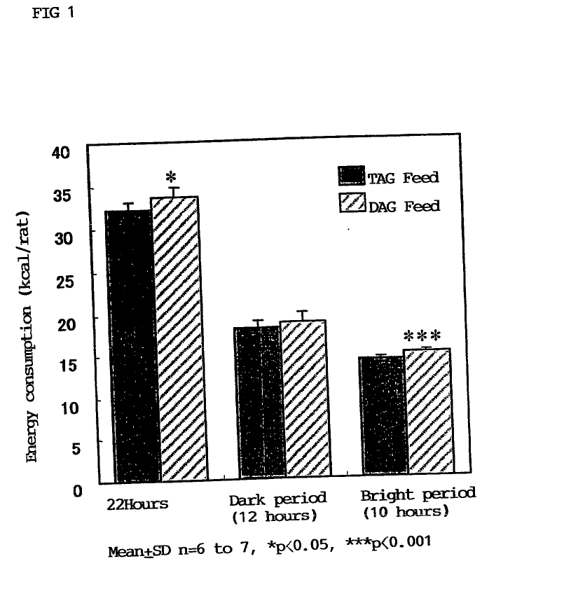

As a result, the DAG group was significantly high in the total energy consumption over 22 hours than the TAG group (p<0.05 vs the TAG group) although there was no difference between the DAG group and the TAG group in the amount of the ingested feed during the 1-week pre-rearing and the measurement of the energy metabolism volumes (22 hours). Especially in an inactive, bright period (7:00 to 17:00), the total energy consumption significantly increased (p<0.001 vs the TAG group) (FIG. 1). As the ingestion of diglycerides led to higher energy consumption than that of triglycerides, it was suggested that diglycerides are more easily burnable as energy. Diet (meal) induced thermogenesis (DIT) was enhanced especially after the ingestion of diglycerides.

### EXAMPLE 5

### Effect of Diglycerides on the Burning of Dietary Lipids

Subsequent to rearing for 4 weeks with a feed which contained diglycerides (DAG) at a concentration of 30% (Table 7), mice (CLEA Japan, Inc.) (n=8 per group) were fasted for 14 hours. Subsequently, triglycerides (TAG) which contained 28% of tripalmitin labeled with ¹³C at the 1-position thereof were administered as an emulsion, the composition of which is shown in Table 8, once by using a feeding tube (“Safeed Fr.3.5”, trade name; product of Terumo Corporation). As a control, mice (n=8) which had been reared for 4 weeks with a feed containing 30% of TAG of the same fatty acid composition were fasted and administered likewise. After the administration of the emulsion, the mice in the respective groups were separately placed in metabolic cages [“METABOLICA” (trade mark), manufactured by Sugiyama-Genki Iriki Co., Ltd.)], and their expirations were caused to be absorbed in portions of a 5 N aqueous solution of sodium hydroxide before the initiation of the experiment and from the 0^{th} hour to 10^{th} hour, from the 10^{th} hour to 24^{th} hour and from the 24^{th} hour to 33^{rd} hours, all after the administration of the emulsion. During the 33 hours for the sampling of the expirations, the DAG feed (the TAG feed for the control) and drink water were given ad libitum. The CO₂ in each expiration sample, which was collected in the aqueous sodium hydroxide solution, was caused to precipitate as CaCO₃ by using calcium chloride and ammonium chloride. The amount of ¹³C contained in the CaCO₃ was determined using a mass spectrometer (“ANCA-SL”, trade name; manufactured by PDZ Europe Ltd.). In this manner, variations in the level of ¹³C—CO₂ in the expiration from the mice in each group were investigated. Further, mice were similarly reared, and were likewise orally administered with triglycerides which contained [1-¹³C]-tripalmitin labeled with ¹³C at the 1-position thereof. Those mice were then fed with the same test feeds, respectively, and were sacrificed 24 hours later or 32 hours later to collect their epididymal fat tissues and mesenteric fat tissues. From each of those organs, lipids were extracted with a 1:2 v/v mixed solvent of methanol and chloroform. The amount of ¹³C in the whole lipids was quantitated, and was presented as a percent accumulation based on the administered amount.

TABLE 7
Compositions of Mouse Feeds

| | TAG feed group (%) | DAG feed group (%) |
|---|---|---|
| TAG | 30 | 0 |
| DAG | 0 | 30 |
| Sucrose | 13 | 13 |
| Cellulose | 4 | 4 |
| Mineral mix | 3.5 | 3.5 |
| Vitamin mix | 1 | 1 |
| Potato starch | 48.5 | 48.5 |
| Total | 100.0 | 100.0 |

DAG: Oil or fat composition B, TAG: Soybean oil

TABLE 8
Composition of emulsion

| | (%) |
|---|---|
| Mixed lipids | 5 |
| Lecithin | 0.2 |
| Albumin | 2 |
| Distilled water | 92.8 |
| Total | 100.0 |

Mixed lipids (with 28% of ¹³C-labeled tripalmitin)

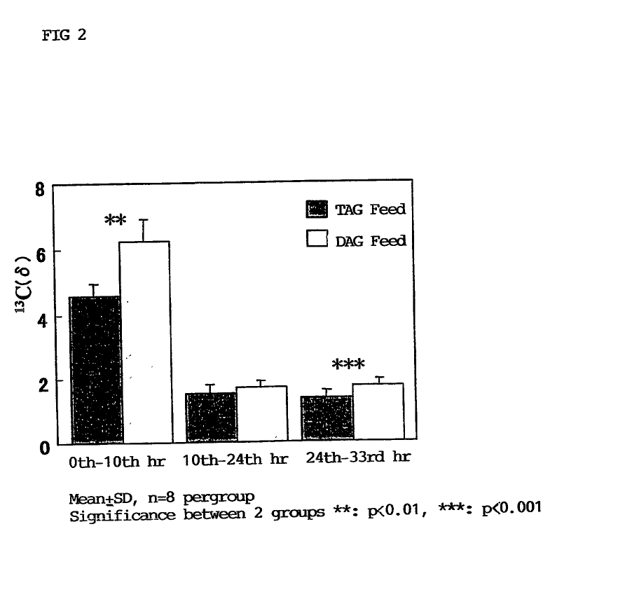

As a result, in each of the DAG administered group and the control group, ¹³C—CO₂ derived from the single-administered lipids was released into the expiration from the 0^{th} hour to 10^{th} hour after the administration of the labeled lipids, and after the 10^{th} hour, its concentration dropped (FIG. 2). Further, the amounts of ¹³C—CO₂ in the expirations from the 0^{th} hour to 10^{th} hour and from the 24^{th} hour to 33^{rd} hour were significantly higher in the TAG feed group than in the DAG feed group although there was no difference in the amount ingested during the expiration sampling time between the DAG group and the TAG group. This clearly indicates that long-term ingestion of diglycerides promotes oxidative degradation (burning) of TAG ingested from other feeds. As body fat accumulation suppressing effect of diglycerides, energy releasing effect associated with burning of dietary lipids subsequent to ingestion of diglycerides was demonstrated.

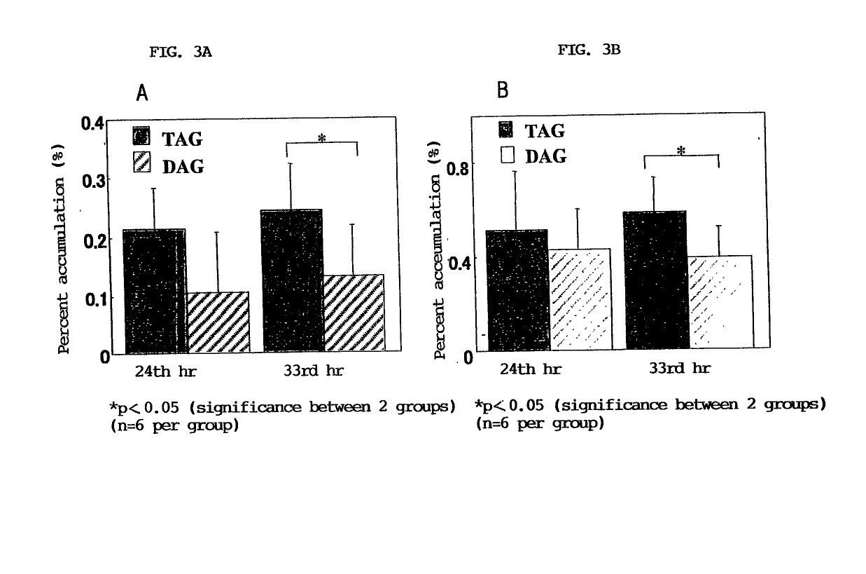

In each of the DAG feed group and the TAG feed group, the percent accumulation of ¹³C in fat was higher in the mesenteric fat (B) than in the epididymal fat (A). On the 33^{rd} hour after the administration of the lipids, the percent accumulations of ¹³C in both the epididymal fat and mesenteric fat were both found to be significantly low values in the DAG feed group than in the TAG feed group (FIG. 3).

From the foregoing, diglycerides have been found to be equipped with effect that, when ingested, they promote burning not only diglycerides but also other dietary lipids to excrete them as an expiration and hence, to suppress their accumulation as body fat.

### EXAMPLE 6

### Remnant-Like Lipoprotein (RLP) Level Lowering Effect

The groups of volunteers relatively high in serum triglyceride level, each consisting of 8 adult male and female subjects, used the above-described oil or fat compositions A to E, respectively, for one month (average ingestion: 10 g/day) in place of edible oils which they had used daily. Blood samples were drawn both before and after the use of the oil or fat compositions A to E, and their serum RLP levels were measured (Table 9).

The serum RLP levels were each quantitated based on the amount of cholesterol in a fraction which had been obtained by conducting fractionation with an anti-apo B-100-anti-apo A1 monoclonal antibody affinity mixed gel.

TABLE 9

| | | |
|---|---|---|
| Oil or fat composition | c/(t + S) | RLP level |

| | | | |
|---|---|---|---|
| Invention | A | 20.1 | 83.1 |
| | B | 11.3 | 86.3 |
| | C | 6.2 | 92.1 |
| | D | NT | 90.3 |
| Comparative | Soybean oil | 5.1 | 103.8 |

Shown in terms of relative value when the initial values were supposed to be 100.
NT: Not tested

As the ingestion of the diglyceride-containing oil or fat compositions A to E was able to lower the serum RLP levels, diglycerides can prevent diseases such as angina pectoris and myocardial infarction.

### EXAMPLE 7

### Serum Leptin Lowering Effect

The groups of volunteers high in body mass index, each consisting of 5 male subjects and 9 female subjects, used the above-described oil or fat compositions A to D, respectively, for one month (average ingestion: 10 g/day) in place of edible oils which they had used daily. Blood samples were drawn both before and after the use of the oil or fat compositions A to D, and their serum leptin levels were measured (Table 10). The leptin levels were quantitated by the method which performs a measurement by using an antibody to human leptin [Clin. Chem., 42, 942 (1996)].

TABLE 10

| | | Relative serum |
|---|---|---|
| Oil or fat composition | | leptin level |
| Invention | A | 32.5 |
| | B | 85.2 |
| | C | 92.0 |
| | D | 90.1 |
| Comparative | Soybean oil | 105.8 |

Shown in terms of relative value when the serum leptin levels before the ingestion were supposed to be 100.

The subjects who ingested the oil or fat composition A were found from CT scan images of their umbilical region that, as the serum leptin level lowered to 82.5% compared with the serum leptin level before the digestion (which was supposed to be 100), the subcutaneous fat area and visceral fat area dropped to 93.9% and 94.4%, respectively, and at the same time, the serum triglyceride level also dropped to 89.0%.

The oil or fat compositions A to D were all excellent in serum leptin level lowering effect.

What is claimed is:

1. A method for activating lipid catabolism in the small intestine epithelium, which comprises administering an effective amount of a diglyceride.

2. The method according to claim 1, wherein 15 to 90 wt. % of constituent fatty acids of said diglyceride comprise ω3 unsaturated fatty acids.

3. The method according to claim 1 or 2, wherein 1,3-diglycerides in said diglyceride amount to at least 50 wt. % of the whole diglyceride.

4. A method for promoting accumulation of fatty acids into the small intestine epithelium, which comprises administering an effective amount of a diglyceride.

5. The method according to claim 4, wherein 15 to 90 wt. % of constituent fatty acids of said diglyceride comprise ω3 unsaturated fatty acids.

6. The method according to claim 4 or 5, wherein 1,3-diglycerides in said diglyceride amount to at least 50 wt. % of the whole diglyceride.

7. A method for inducing expression of a small intestine lipid metabolic gene, which comprises administering an effective amount of a diglyceride.

8. The method according to claim 7, wherein 15 to 90 wt. % of constituent fatty acids of said diglyceride comprise ω3 unsaturated fatty acids.

9. The method according to claim 7 or 8, wherein 1,3-diglycerides in said diglyceride amount to at least 50 wt. % of the whole diglyceride.

10. A method for suppressing synthesis of a triglyceride in the small intestine epithelium, which comprises administering an effective amount of a diglyceride.

11. The method according to claim 10, wherein 15 to 90 wt. % of constituent fatty acids of said diglyceride comprise ω3 unsaturated fatty acids.

12. The method according to claim 10 or 11, wherein 1,3-diglycerides in said diglyceride amount to at least 50 wt. % of the whole diglyceride.

13. A method for promoting energy consumption, which comprises administering an effective amount of a diglyceride.

14. The method according to claim 10, wherein 15 to 90 wt. % of constituent fatty acids of said diglyceride comprise ω3 unsaturated fatty acids.

15. The method according to claim 13 or 14, wherein 1,3-diglycerides in said diglyceride amount to at least 50 wt. % of the whole diglyceride.

16. A method for lowering a serum RLP level, which comprises administering an effective amount of a diglyceride.

17. The method according to claim 16, wherein 15 to 90 wt. % of constituent fatty acids of said diglyceride comprise ω3 unsaturated fatty acids.

18. The method according to claim 16 or 17, wherein 1,3-diglycerides in said diglyceride amount to at least 50 wt. % of the whole diglyceride.

19. A method for lowering a serum leptin level, which comprises administering an effective amount of a diglyceride.

20. The method according to claim 19, wherein 15 to 90 wt. % of constituent fatty acids of said diglyceride comprise ω3 unsaturated fatty acids.

21. The method according to claim 19 or 20, wherein 1,3-diglycerides in said diglyceride amount to at least 50 wt. % of the whole diglyceride.
